(19) United States
(12) Patent Application Publication
(10) Pub. No.: US 2004/0179664 A1
(43) Pub. Date: Sep. 16, 2004

(54) Processing apparatus for acquired signals

(76) Inventors: Harry Louis Platt, Eastgardens (AU); Allan Michael Shell, Eastgardens (AU); Vladimir Jankov, Eastgardens (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/451,451

(22) PCT Filed: Dec. 21, 2001
(86) PCT No.: PCT/AU01/01651

(30) Foreign Application Priority Data
Dec. 22, 2000 (AU) PR 2297

(51) Int. Cl. A61B 7/04; H04M 11/00; H04B 1/00

(52) U.S. Cl. 379/106.02; 379/110.01; 381/67

(57) ABSTRACT

A transtelephonic process and storage apparatus (10) for use with a blood sugar monitor or like standalone monitoring apparatus having an output port to download acquired digital data signals is disclosed. The apparatus (10) has a memory (18), a controlling (14), a transducer (17) and an output (12), wherein the downloaded acquired digital is converted into audio transmission signals by the transducer (17), and the audio transmission signal is stored in memory (18) for delayed transmission over a telephone network.

The present invention relates to physiological monitoring of patients and, in particular, to physiological signal acquisition apparatus which provides recorded data transmission over a telephone network to a remote location where it is decoded and analysed.

### BACKGROUND TO THE INVENTION

It would be advantageous to provide a blood sugar measurement apparatus which provides delayed transmission of recorded blood sugar data to a remote monitoring location, such that it can be collated and analysed and reported to a physician, by providing a digital data acquiring apparatus which then provides the acquired digital signal to a processing unit for processing and storage into an analog audio signal suitable for transmission over a telephone network.

Furthermore, it would be advantageous for the processing unit to be applicable to process digital signals from standalone acquiring apparatus which is used to acquire and display the data in real time.

### OBJECT OF THE INVENTION

It is an object of the present invention to provide a processing apparatus which substantially provides the above mentioned advantages. At the very least, the object of the invention is to provide an alternative to known monitoring and display apparatus.

### DISCLOSURE OF THE INVENTION

According to one aspect of the present invention there is disclosed a transtelephonic process and storage apparatus for use with a blood sugar monitor or like standalone monitoring apparatus having an output port to download acquired digital data signals, said apparatus having memory means, a control means, a transducer means and output means, wherein said downloaded acquired digital is converted into audio transmission signals by said transducer means, said audio transmission signal being stored in said memory means for delayed transmission over a telephone network and wherein said control means controlling the operation of said apparatus.

Preferably the transducer means converts the data into signals which are sent to a speaker output as audio signals which are preferably transmitted over the telephone network to a remote receiving station which decodes and analyses the received audio signals.

Preferably the apparatus has switching means to activate the transmission of the. audio signals from said memory means.

### BRIEF DESCRIPTION OF THE DRAWINGS

The present invention will be now be described with reference to the accompanying drawings in which:

FIG. 1 is a schematic block diagram of the apparatus of the present invention.

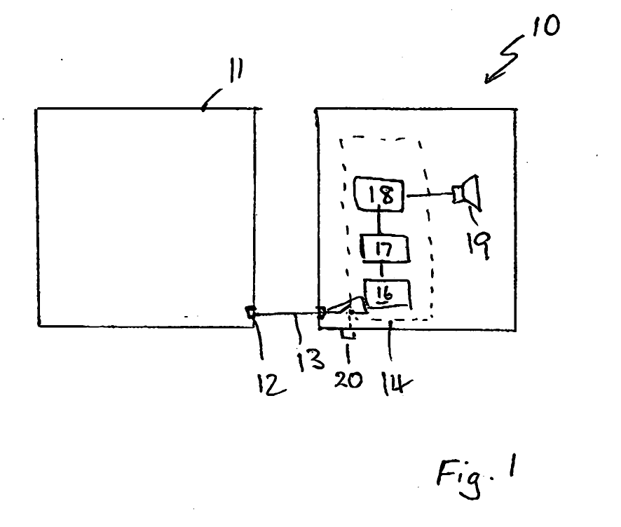

### BEST MODE OF CARRYING OUT THE INVENTION

A processing apparatus 10 is illustrated in the drawing. The apparatus 10 is used to process acquired digital data downloaded from a blood sugar monitor 11 which has an output port 12. The apparatus 10 is connected to the output port 12 by a cable 13. The monitor 11 is of the type which is used to display the results of a physiological test but also has the output port 12 whereby the acquired digital data signals can be downloaded to computers or the like. Naturally, the apparatus 10 can be used with any similar monitor which has a suitable output port 12.

The apparatus 11 has a controller 14, including a microprocessor 15 and amplifier 16, a transtelephonic hardware/firmware converter 17, memory 18 and a speaker 19. The acquired digital data is processed by the apparatus 10 and converted into the appropriate audio signals by the transtelephonic hardware/firmware converter 17 and then stored into the memory 18. A transmit button 20 located on the body of the apparatus 10 is activated by which the controller 14 arranges for the audio signals to be transmitted by the speaker 19. These audio signals can then be transmitted via an acoustically coupled telephone connection over the telephone network to a remote receiving station. The data can be either transmitted in real time or can be sent at a later time, after being retrieved from the memory.

The remote receiving station preferably automatically decodes and analyses the acoustically coupled data and creates a report for a physician as required.

Technique of encoding and decoding of analogue and digital data can be implemented in different forms such as frequency or phase modulation, dual tone multi-frequency etc.

The foregoing describes only one embodiment of the present invention, and modifications obvious to those skilled in the art can be made thereto without departing from the scope of the present invention.

1. A transtelephonic process and storage apparatus for use with a blood sugar monitor or like standalone monitoring apparatus having an output port to download acquired digital data signals, said apparatus having memory means, a control means, a transducer means and output means, wherein said downloaded acquired digital is converted into audio transmission signals by said transducer means, said audio transmission signal being stored in said memory means for delayed transmission over a telephone network and wherein said control means controlling the operation of said apparatus.

2. The apparatus according to claim 1, wherein the transducer means converts the data into signals which are sent to a speaker output as audio signals.

3. The apparatus according to claim 2, wherein the audio signals are transmitted over the telephone network to a remote receiving station which decodes and analyses the received audio signals.

4. The apparatus according to claim 3, wherein the apparatus has switching means to activate the transmission of the audio signals from said memory means.
